(19) United States
(12) Patent Application Publication
(10) Pub. No.: US 2022/0135050 A1
(43) Pub. Date: May 5, 2022

(54) EYE-GAZE INPUT APPARATUS

(71) Applicant: Panasonic Intellectual Property Management Co., Ltd., Osaka (JP)

(72) Inventors: Asuka EGASHIRA, OSAKA FU (JP); Seigo TAKADA, OSAKA FU (JP); Taiki TAKAAZE, OSAKA FU (JP); Kenya MATSUSHITA, KYOTO FU (JP); Hiroshi TAKEKAWA, OSAKA FU (JP)

(73) Assignee: Panasonic Intellectual Property Management Co., Ltd., Osaka (JP)

(21) Appl. No.: 17/512,037

(22) Filed: Oct. 27, 2021

(30) Foreign Application Priority Data
Oct. 29, 2020 (JP) 2020-181907

(51) Int. Cl. B60W 40/08; G06K 9/00; B60W 50/14; G06F 3/01

(52) U.S. Cl. CPC B60W 40/08; G06K 9/00845; B60W 2050/146; G06F 3/017; B60W 2040/0863; B60W 50/14

(57) ABSTRACT

An eye-gaze input apparatus includes a hardware processor functioning as an eye-gaze determination unit, an input/output processing unit, and a careless-state determination unit. The eye-gaze determination unit determines a first input by an eye gaze of a user with respect to first images of input elements displayed in a display region set in front of a windshield and a driver seat of a vehicle. The careless-state determination unit determines whether the user is in a careless state. The input/output processing unit confirms the first input in a case where the eye-gaze determination unit determines that there is the first input on any of the one or more first images and the careless-state determination unit determines that the user is not in the careless state, and does not confirm the first input in a case where the careless-state determination unit determines that the user is in the careless state.

### CROSS-REFERENCE TO RELATED APPLICATIONS

This application is based upon and claims the benefit of priority from Japanese Patent Application No. 2020-181907, filed on Oct. 29, 2020, the entire contents of which are incorporated herein by reference.

### FIELD

The present disclosure relates to an eye-gaze input apparatus.

### BACKGROUND

Japanese Patent Application Laid-open No. 2014-218199 discloses an eye-gaze input apparatus that selects an input element by an eye gaze. In this eye-gaze input apparatus, images of input elements are displayed, by a head-up display device, in a display region set in front of a driver seat of a vehicle. A gaze point of a user viewing one of the images displayed in the display region is detected by an eye-gaze detection device installed in front of the driver seat, and thereby an eye-gaze input on the head-up display device is performed.

According to the eye-gaze input apparatus described above, an eye-gaze input by a user is treated as valid even in a case where the user is in a careless state. However, such an eye-gaze input may not conform to intention of the user, and this may cause an erroneous output.

Therefore, it is desired to restrain such an unintended eye-gaze input performing by a user who is in a careless state.

### SUMMARY

An eye-gaze input apparatus according to an embodiment includes a hardware processor configured to function as an eye-gaze determination unit, an input/output processing unit, and a careless-state determination unit. The eye-gaze determination unit determines a first input by an eye gaze of a user with respect to one or more first images of one or more input elements, the one or more first images being displayed in a display region set in front of a windshield and in front of a driver seat of a vehicle. The input/output processing unit outputs a command to an external device corresponding to an input element on which the first input is detected among the one or more input elements. The careless-state determination unit determines, based on a state of the user, whether the user is in a careless state. The input/output processing unit confirms the first input with respect to the input element on which the first input is detected in a case where the eye-gaze determination unit determines that there is the first input on any of the one or more first images and the careless-state determination unit determines that the user is not in the careless state. The input/output processing unit does not confirm the first input with respect to the input element n a case where the careless-state determination unit determines that the user is in the careless state.

### BRIEF DESCRIPTION OF THE DRAWINGS

FIG. 1 is a block diagram illustrating an example of a configuration of an eye-gaze input apparatus in a first embodiment;

FIG. 2 is a view illustrating an example of use of an HUD device in the first embodiment;

FIG. 3 is a view illustrating an application example of the eye-gaze input apparatus in the first embodiment;

FIG. 4A is a view illustrating an example of an input element displayed in a display region by the HUD device in the first embodiment;

FIG. 4B is a view illustrating another example of an input element displayed in the display region by the HUD device in the first embodiment;

FIG. 5 is a flowchart illustrating an example of eye-gaze input processing by the eye-gaze input apparatus in the first embodiment;

FIG. 6 is a block diagram illustrating an example of a configuration of an eye-gaze input apparatus in a second embodiment; and

FIG. 7 is a flowchart illustrating an example of eye-gaze input processing by the eye-gaze input apparatus in the second embodiment.

### DETAILED DESCRIPTION

In the following, embodiments will be described in detail with reference to the drawings as appropriate. However, unnecessarily detailed description may be omitted. For example, a detailed description of a well-known matter and a repeated description of substantially the same configuration may be omitted. This is to avoid unnecessary redundancy of the following description and to facilitate understanding of those skilled in the art.

Note that the accompanying drawings and the following description are provided for those skilled in the art to fully understand the present disclosure, and are not intended to limit subject matter described in claims.

### First Embodiment

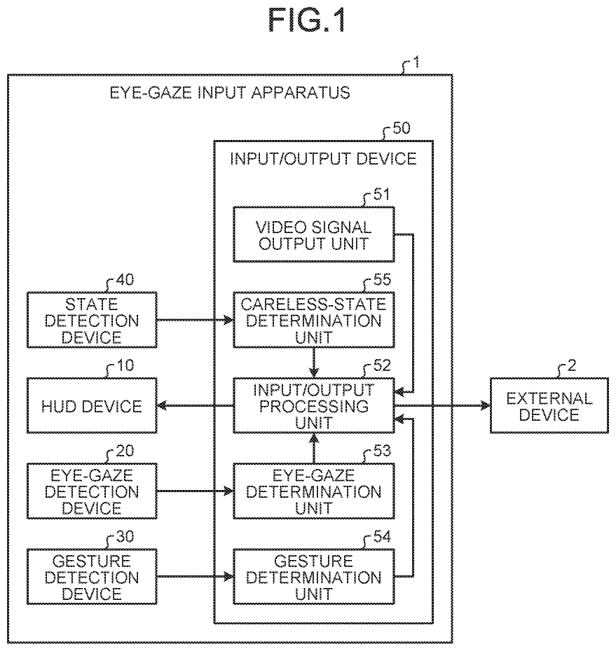

In the following, the first embodiment will be described with reference to FIGS. 1 to 5.

Configuration

First, a configuration of an eye-gaze input apparatus according to the present embodiment will be described with reference to FIGS. 1 to 3.

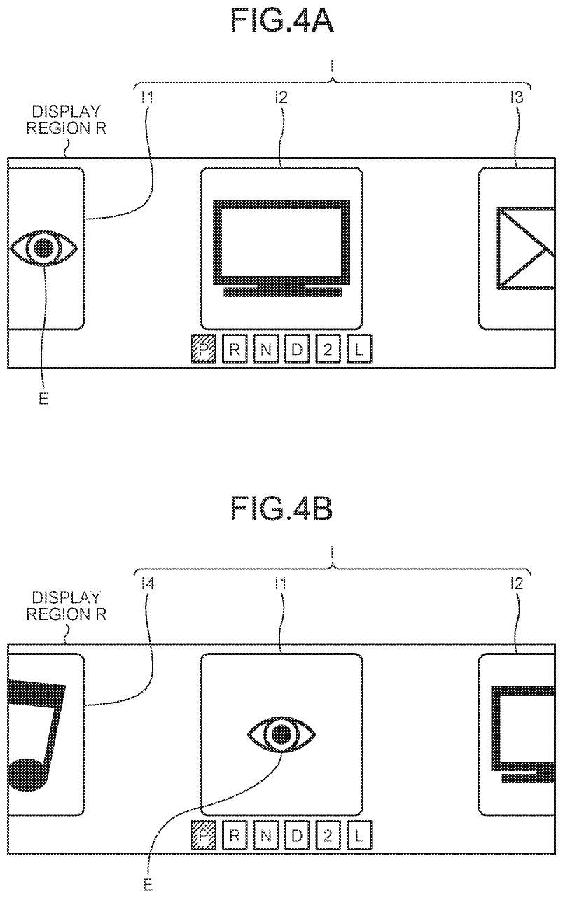

As illustrated in FIG. 1, an eye-gaze input apparatus 1 according to the present embodiment includes a head-up display (HUD) device 10, an eye-gaze detection device 20, a gesture detection device 30, a state detection device 40, and an input/output device 50. The eye-gaze input apparatus 1 detects that a user 200 is gazing at an input element displayed in a display region R (illustrated in FIGS. 4A and 4B) of the HUD device 10, and confirms an input of a selected input element on condition that a predetermined gesture by the user 200 is detected.

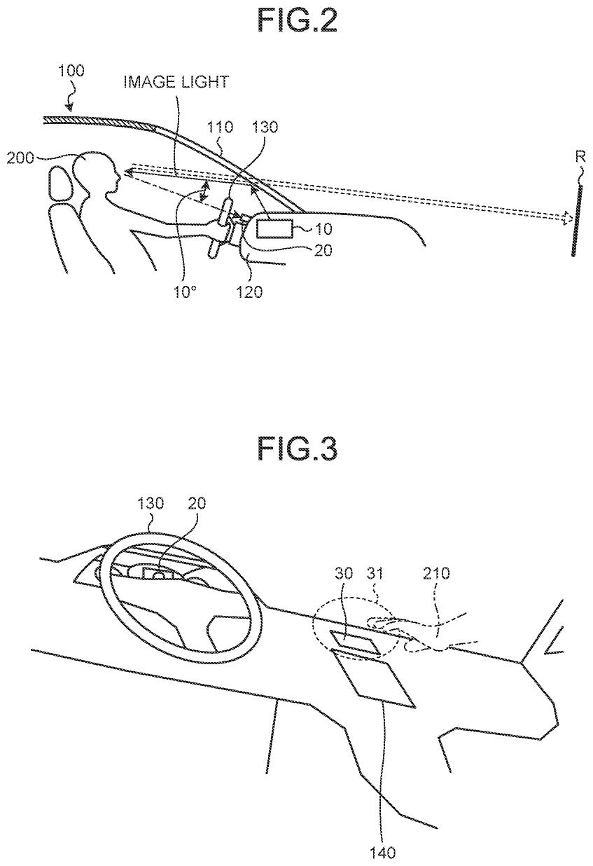

As illustrated in FIG. 2, the HUD device 10 is built in a dashboard 120 of a vehicle 100. The HUD device 10 projects, onto a windshield 110 of the vehicle 100, image light output by the input/output device 50, which includes an image of the input element. As a result, the image light is reflected by the windshield 110 and travels toward the user 200 who is a driver of the vehicle 100, for example. Consequently, the user 200 visually recognizes the image, which is indicated by the image light through the windshield 110, as a virtual image existing in a virtual display region R set in front of the vehicle 100. That is, the HUD device 10 causes the user 200 to visually recognize, as the virtual image, the image indicated by the image light. Note that the image of the input element displayed in the display region R will be described later in detail. The windshield 110 is an example of a display medium. In the present embodiment, the display medium is the windshield 110. However, in a case where the vehicle 100 includes a combiner, the HUD device 10 may project image light onto the combiner as a display medium. An image that is an input element is an example of a “first image” in the present embodiment.

The eye-gaze detection device 20 is a device that is installed in front of a driver seat to detect a gaze point of the user 200 seated in the driver seat. Here, the gaze point of the user 200 corresponds to a “first input” in claims. Specifically, the eye-gaze detection device 20 is a camera that is installed at the back of a steering wheel 130 of the vehicle 100 as viewed from the user 200 and that photographs eyes of the user 200 irradiated with invisible near-infrared light. For detecting the gaze point of the user 200, it is preferable that the eye-gaze detection device 20 is installed within 30° above, below, left, and right of the gaze point of the user 200. In the present embodiment, for detecting a gaze point of the user 200 on the display region R, the eye-gaze detection device 20 is installed on the dashboard 120 at a position of 10° below the center of the display region R as viewed from the user 200.

As illustrated in FIG. 3, the gesture detection device 30 is arranged, for example, above a screen of a car navigation device 140. In FIG. 3, a gesture detection region 31 of the gesture detection device 30 is indicated by a dashed circle. The gesture detection device 30 detects a non-contact gesture performed by the user 200 in the gesture detection region 31. Here, the gesture is an example of a “second input” by the user 200. The gesture detection device 30 is, for example, a capacitive gesture sensor. The capacitive gesture sensor includes a plurality of electrodes to measure a temporal change in capacitance of each electrode. The capacitive gesture sensor estimates that a gesture is performed in response to determining that the temporal change of the capacitance is similar to a change defined in advance as a gesture.

Note that the gesture detection device 30 may be installed on an instrument panel or the like below the eye-gaze detection device 20. Additionally, the gesture detection device 30 may be an infrared or sonic gesture sensor, a gesture sensor using an imaging device, or the like.

The state detection device 40 includes various sensors installed in the vehicle 100 to detect a state of the user 200. As an example, the state detection device 40 is a camera that photographs a face of the user 200. The state of the user 200 indicates a movement of an eye gaze, an expression, and the like of the user 200. This camera is installed at the back of the steering wheel 130 of the vehicle 100 as viewed from the user 200. Note that this camera may be one shared as the above-described camera functioning as the eye-gaze detection device 20, or may be provided separately from the camera as the eye-gaze detection device 20. Also, the state detection device 40 may be a heartbeat detection device that detects a heartbeat of the user 200, and a state of the user 200 in this case indicates a heart rate, intensity of a heartbeat, and the like of the user 200. This heartbeat detection device may be a contact sensor provided on the steering wheel 130 or a seat of the vehicle 100 or attached to a body of the user 200, or may be a non-contact sensor such as a camera that detects a change in a complexion of the user 200 which change corresponds to a pulse wave.

The input/output device 50 includes a video signal output unit 51, an input/output processing unit 52, an eye-gaze determination unit 53, a gesture determination unit 54, and a careless-state determination unit 55.

The video signal output unit 51 outputs, to the input/output processing unit 52, a video signal to be displayed in the display region R.

The eye-gaze determination unit 53 determines a first input by an eye gaze of the user 200 with respect to one or more images of one or more of input elements displayed by the HUD device 10. More specifically, on the basis of a detection result acquired from the eye-gaze detection device 20, the eye-gaze determination unit 53 determines an image out of the one or more images, on which the first input is performed. For example, the eye-gaze determination unit 53 detects, from an image of an eye of the user 200 photographed by the eye-gaze detection device 20, a pupil(s) whose position changes depending on a gaze direction and the corneal reflection that is not affected by the gaze direction, and then detects a gaze point (first input) from a positional relation between the pupil(s) and the corneal reflection. Then, the eye-gaze determination unit 53 outputs coordinates of the detected gaze point to the input/output processing unit 52.

The gesture determination unit 54 outputs an input confirmation signal to the input/output processing unit 52 on condition that a specific gesture (second input) of the user 200, such as a gesture of waving a hand is detected by the gesture detection device 30.

The second input is not limited to a specific gesture although it is treated as the second input in the present embodiment. For example, an eye gaze, blink, voice, a touch, or the like by the user 200 may be detected as the second input. That is, the gesture detection device 30 is an example of an “input detection device” that detects the second input, and a different device such as a camera, a microphone, a touch panel, or a button can be used as the input detection device as long as any input by the user 200 can be detected. The gesture determination unit 54 is an example of an “input determination unit” that determines the second input. The input determination unit is capable of determining a predetermined eye gaze, blink, voice, touch, or the like by the user 200, based on an input by the input detection device.

The careless-state determination unit 55 determines, based on a state of the user 200, whether or not the user 200 is in a careless state. More specifically, the careless-state determination unit 55 acquires, from the state detection device 40, a state of the user 200 detected by the state detection device 40, and determines whether the user 200 is in the careless state based on the acquired state of the user 200. The careless state refers to, for example, a state where the user 200 lacks concentration or attention.

The careless-state determination unit 55 determines whether the user 200 is in the careless state on the basis of, for example, a deviation between an object located in a gaze direction of the user 200 and a focal position of the user 200. Specifically, the careless-state determination unit 55 calculates respective gaze directions of a right eye and a left eye of the user 200 on the basis of a face image of the user 200, and calculates the focal position of the user 200 therefrom. Subsequently, the careless-state determination unit 55 calculates a position of the object included in a front image and located in the gaze direction of the user 200. Then, the careless-state determination unit 55 determines that the user 200 is in the careless state in a case where a distance between the position of the target object and the focal position is equal to or longer than a predetermined threshold.

The careless-state determination unit 55 can determine whether the user 200 is in the careless state on the basis of a change amount of the orientation of a face of the user 200. Specifically, the careless-state determination unit 55 first determines whether a speed variation and a steering angle variation of the vehicle 100 per given time are each equal to or smaller than a predetermined threshold. The careless-state determination unit 55 determines that the vehicle 100 is monotonously traveling in response to determining that the variations are each equal to or smaller than the predetermined threshold. Then, upon determining that the vehicle 100 is monotonously traveling, the careless-state determination unit 55 further determines whether the change amount of the face orientation of the user 200 per given time is equal to or smaller than a predetermined threshold. In response to determining that the change amount is equal to or smaller than the predetermined threshold, the careless-state determination unit 55 finally determines that the user 200 is in the careless state.

Note that a determination method of the careless state by the careless-state determination unit 55 is not limited to the above-described contents. Various methods, such as determination based on an expression or a heartbeat of the user 200, can be employed.

The input/output processing unit 52 outputs, to the HUD device 10, the video signal transmitted from the video signal output unit 51. Also, the input/output processing unit 52 determines an input element selected by the user 200 based on the video signal transmitted from the video signal output unit 51 and coordinates of the gaze point transmitted from the eye-gaze determination unit 53. Then, the input/output processing unit 52 outputs a command to an external device corresponding to the input element. More specifically, upon receiving the input confirmation signal from the gesture determination unit 54, the input/output processing unit 52 confirms an input of the input element selected by the user 200 and outputs a command to an external device 2 corresponding to the input element.

Also, in a case where the eye-gaze determination unit 53 determines that the first input is performed on any of the images of the input elements and the careless-state determination unit 55 determines that the user 200 is not in the careless state, the input/output processing unit 52 confirms the input of the input element on the basis of the first input. Then, the input/output processing unit 52 outputs a command to the external device 2 corresponding to the input element.

On the other hand, in a case where the careless-state determination unit 55 determines that the user 200 is in the careless state, the input/output processing unit 52 does not confirm the input of the input element based on the first input by the user 200. In the present embodiment, in the case where the careless-state determination unit 55 determines that the user 200 is in the careless state, the input/output processing unit 52 does not output a command to the external device 2 corresponding to the input element regardless of whether the input confirmation signal is received from the gesture determination unit 54 thereafter.

The input/output device 50 includes an electronic control unit (ECU) containing a central processing unit (CPU), a read only memory (ROM), a random access memory (RAM), an input/output port (I/O), and the like. The CPU (an example of the hardware processor) in the input/output device 50 functions as the above-described video signal output unit 51, input/output processing unit 52, eye-gaze determination unit 53, and gesture determination unit 54 by executing one or more computer programs stored in the ROM or the like. Alternatively, the input/output device 50 may be configured by a plurality of CPUs functioning as the video signal output unit 51, the input/output processing unit 52, the eye-gaze determination unit 53, and the gesture determination unit 54.

Operation

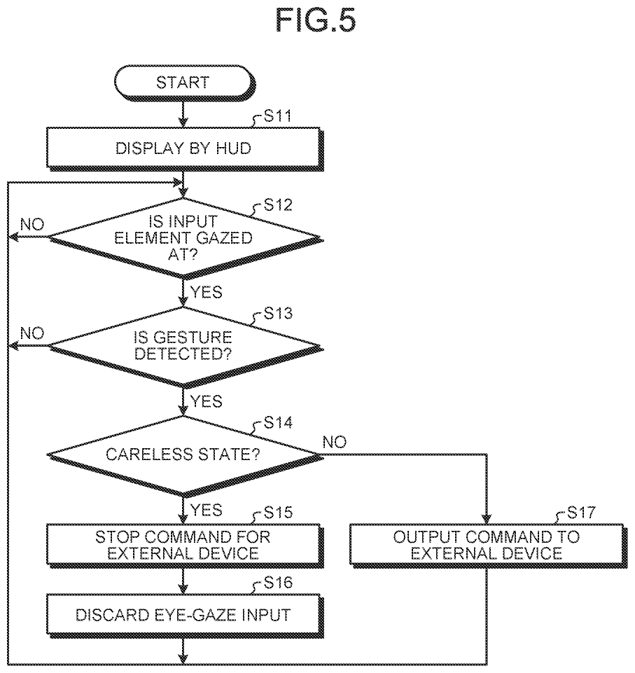

Next, an operation of the eye-gaze input apparatus according to the present embodiment will be described with reference to FIGS. 4A to 5.

Input elements displayed in the display region R by the HUD device 10 will be described with reference to FIGS. 4A and 4B. In FIGS. 4A and 4B, an eye symbol E represents a gaze point of the user 200 although not actually displayed in the display region R. As illustrated in FIG. 4A, the HUD device 10 displays images I1, I2, and I3 of input elements in a row within the display region R. Note that images such as the images I1 to I3 of the input elements are collectively referred to as images I of the input elements in the following. In the present embodiment, the number of images I of the input elements to be displayed is three. The HUD device 10 does not display outer portions of images I of input elements displayed at both left and right ends of the display region R. That is, a left portion of the image I1 of the input element displayed at the left end, and a right portion of the image I3 of the input element displayed at the right end are not displayed. In this manner, it is indicated that the images I of the input elements are not limited to the three displayed in the display region R but are continued to both left and right sides of the display region R. Also, the HUD device 10 displays, on a lower side at the center of the display region R, a transmission position of a transmission of the vehicle 100. In the present embodiment, for safety of traveling, an input of an input element by the user 200 is enabled only when the vehicle 100 is stopped. Since the transmission position of the transmission of the vehicle 100 is displayed by the HUD device 10, the user 200 understands that the eye-gaze input is possible in a case where the transmission position displayed in the display region R is parking.

In FIG. 4A, the user 200 gazes at and selects the image I1 of the input element arranged at the left end. Here, the gaze point of the user 200 is located on an image different from the image I2 of the input element at the center of the row. In this case, the input on the image I1 of the input element, at which the gaze point is located, is not confirmed even when the user 200 performs a gesture in this state. When the gaze point of the user 200 is on an image different from the image I2 of the input element at the center of the row, the HUD device 10 moves the image I1 of the input element, at which the gaze point of the user 200 is located, to the center of the row and performs a display thereof in the display region R, as illustrated in FIG. 4B. That is, the HUD device 10 displays the images I of the input elements in the display region R while shifting them rightward by one from the state of FIG. 4A. As a result, as shown in FIG. 4B, the image I3 of the input element displayed on the right side in FIG. 4A is not displayed, and an image I4 of an input element that was not displayed in the state of FIG. 4A is displayed at the left end in FIG. 4B. In this state, when the user 200 performs a predetermined gesture such as waving a hand 210 while gazing at the image I1 of the input element located at the center, the input of the input element at which the gaze point of the user 200 is located is confirmed by the input/output processing unit 52.

Next, a processing operation of the eye-gaze input apparatus 1 will be described with reference to FIG. 5.

First, in Step S11, the HUD device 10 displays images of input elements on the windshield 110. The HUD device 10 is activated when, for example, an ignition of the vehicle 100 is turned on, and then starts projection of the image onto the windshield 110. Note that, not only the HUD device 10 but also the eye-gaze detection device 20, the gesture detection device 30, and the state detection device 40 may be activated when the ignition of the vehicle 100 is turned on. Also, contents of the image displayed by the HUD device 10 may be appropriately updated regardless of a flowchart of eye-gaze input processing in FIG. 5.

Then, in Step S12, the eye-gaze determination unit 53 determines whether the user 200 is gazing at the image of the input element. Specifically, the eye-gaze detection device 20 photographs the eyes of the user 200 and outputs an image thereof to the eye-gaze determination unit 53. Then, the eye-gaze determination unit 53 detects a gaze point of the user 200 based on the image of the eyes of the user 200 photographed by the eye-gaze detection device 20, and determines whether the eye gaze of the user 200 is directed to the image of the input element on the basis of the detected gaze point. In response to determining that the user 200 is gazing at the image of the input element (YES in Step S12), the eye-gaze determination unit 53 gives, to the input/output processing unit 52, information related to the input element corresponding to the detected gaze point. Then, the processing proceeds to Step S13.

In Step S13, the gesture determination unit 54 determines whether the user 200 is performing a predetermined gesture. Specifically, the gesture detection device 30 detects a gesture of a movement of the hand 210 of the user 200, such as holding the hand over the gesture detection device 30, and outputs information on the detected gesture to the gesture determination unit 54. Then, the gesture determination unit 54 determines whether the gesture output from the gesture detection device 30 is a predetermined gesture. The predetermined gesture is, for example, a gesture of waving a hand. That is, when the gesture output from the gesture detection device 30 is the predetermined gesture such as waving the hand, the gesture determination unit 54 determines that the user 200 is performing the predetermined gesture. In response to determining that the user 200 is performing the predetermined gesture (YES in Step S13), the gesture determination unit 54 gives, to the input/output processing unit 52, information representing that the user 200 is performing the predetermined gesture. Then, the processing proceeds to Step S14.

In Step S14, the careless-state determination unit 55 determines whether the user 200 is in a careless state. Specifically, the state detection device 40 detects a state of the user 200 by a camera or the like functioning as the state detection device 40, and outputs the detected state of the user 200 to the careless-state determination unit 55. Then, the careless-state determination unit 55 determines whether the user 200 is in the careless state on the basis of the state of the user 200 output from the state detection device 40. In response to determining that the user 200 is in the careless state (YES in Step S14), the careless-state determination unit 55 gives, to the input/output processing unit 52, information representing that the user 200 is in the careless state. Then, the processing proceeds to Step S15.

Note that, in the flowchart illustrated in FIG. 5, in a case where the eye-gaze determination unit 53 determines in Step S12 that the user 200 is gazing at the image of the input element, that is, in a case where the first input on the input element is performed by the eye gaze of the user 200, the processing proceeds to Step S14 only in a case of YES in Step S13. However, the flow of the processing is not limited to this. For example, in a case of YES in Step S12, the processing may proceed to Step S14 without execution of Step S13.

In Step S15, the careless-state determination unit 55 gives, to the input/output processing unit 52, information representing that the user 200 is in the careless state, and the input/output processing unit 52 stops outputting a command to the external device 2. Then, the input/output processing unit 52 discards the information from the eye-gaze determination unit 53 in Step S12, which is related to the input element corresponding to the gaze point of the user 200 (Step S16). Note that the input/output processing unit 52 may stop receiving the input of the gaze point from the eye-gaze detection device 20 to the input/output processing unit 52 instead of or in addition to discarding the information related to the input element corresponding to the gaze point of the user 200. Then, the processing returns to Step S12, and the processing in and after Step S12 is executed.

On the other hand, in response to determining that the user 200 is not in the careless state (NO in Step S14), the careless-state determination unit 55 gives, to the input/output processing unit 52, information representing that the user 200 is not in the careless state, and the processing proceeds to Step S17. The input/output processing unit 52 outputs a command to the external device 2 corresponding to the input element selected by the user 200 (Step S17). Then, the processing returns to Step S12, and the processing in and after Step S12 is executed.

Also, in response to determining that the user 200 is not gazing at the image of the input element (NO in Step S12), the eye-gaze determination unit 53 gives, to the input/output processing unit 52, information representing that the user 200 is not gazing at the image of the input element. Then, the processing in and after Step S12 is executed again.

In response to determining that the user 200 is not performing the predetermined gesture (NO in Step S13), the gesture determination unit 54 gives, to the input/output processing unit 52, information representing that the user 200 is not performing the predetermined gesture. Then, the processing returns to Step S12, and the processing in and after Step S12 is executed.

Effects

As described above, the eye-gaze input apparatus 1 according to the present embodiment detects that the user 200 is gazing at an input element displayed in the display region R by the HUD device 10, and outputs a command to the external device 2 corresponding to the input element selected with an eye gaze by the user 200 on condition that the user 200 is not in a careless state.

As a result, when the user 200 is determined to be in the careless state, the selection by the eye gaze of the user 200 is not treated as valid, so that an unintended eye-gaze input by the user 200 can be restrained. In such a case where the user 200 is in the careless state, there may be a situation where the user 200 is unintentionally gazing at a point. If such an eye-gaze input is treated as valid, an input is performed without conforming to intention of the user 200. The eye-gaze input apparatus 1 of the present embodiment is capable of solving such a problem and realizing an eye-gaze input conforming to intention of the user 200.

Also, according to the present embodiment, the eye-gaze input apparatus 1 detects that the user 200 is gazing at an input element, which is displayed in the display region R by the HUD device 10, and further outputs a command to the external device 2, which corresponds to the input element selected with the eye gaze by the user 200, in a case of detecting a predetermined gesture on condition that the user 200 is not in the careless state. Thus, according to the eye-gaze input apparatus 1 of the present embodiment, it is possible to reduce confirmation of an unintended input by the eye gaze of the user 200.

Also, according to the present embodiment, the HUD device 10 displays an image of an input element in the virtual display region R set in front of the driver seat of the vehicle 100, and the eye-gaze detection device 20 installed in front of the driver seat detects a gaze point of the user 200 viewing the image displayed in the display region R. By using the HUD device 10, it is possible to provide the eye-gaze detection device 20 and the display region R in front of the user 200 in a limited space in the vehicle 100. Also, since left and right viewing angles of the image displayed by the HUD device 10 are relatively narrow, the user 200 views the display region R from the front, and a head of the user 200 is substantially fixed to the front. Thus, the eye-gaze detection device 20 can almost always detect the gaze point of the user 200 from the front of the user 200. Thus, by combining the HUD device 10 and the eye-gaze detection device 20, it is possible to appropriately arrange the eye-gaze detection device 20 and the display region R and to highly accurately detect the gaze point of the user 200.

Furthermore, the eye-gaze detection device 20 is set in a predetermined range below the display region R, whereby the gaze point of the user 200 viewing the display region R enters a detection range of the eye-gaze detection device 20. Thus, the gaze point of the user 200 viewing the display region R can be securely detected.

Also, by displaying images of input elements in a row in the display region R and not displaying outer portions of images of input elements displayed at the ends of the row, it is possible to make the images of the input elements appear to be continued to the outside of the display region R. That is, it is possible to cause the user 200 to recognize that there is an image of an input element that is not displayed in the display region R.

Furthermore, by performing a gesture in a state where an input element is selected by an eye gaze, the user 200 can confirm an input of the selected input element. Thus, an image of an input element is displayed in the virtual display region R, and the user 200 can confirm an input of the input element without touching a touch panel, a button, or the like.

Then, in a case where the image of the input element selected with the eye gaze by the user 200 is not at the center, the image of the selected input element is moved to the center. After the image of the input element is moved, the user 200 can confirm the input of the input element by performing a gesture while gazing at the image of the input element.

Furthermore, by limiting the eye-gaze input by the user 200 to the time when the vehicle 100 is stopped, it is possible to restrain a malfunction due to an erroneous input during traveling. Also, since the transmission position of the vehicle 100 is displayed in the display region R, the user 200 can recognize whether the eye-gaze input is possible.

### Second Embodiment

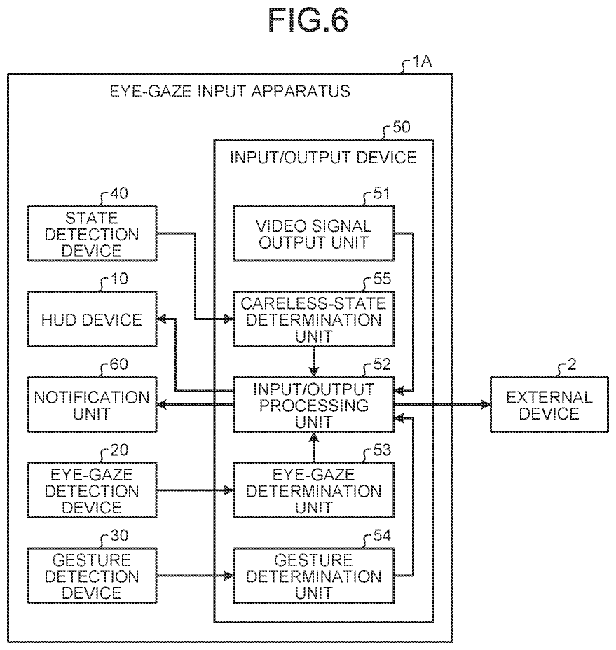

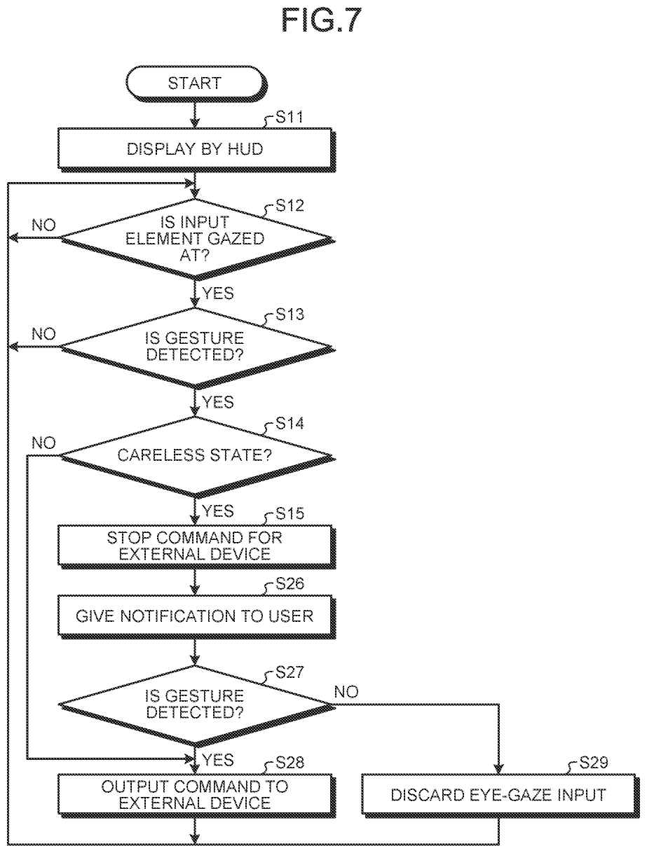

Next, the second embodiment will be described with reference to FIGS. 6 and 7 with a focus on portions different from the first embodiment. As illustrated in FIG. 6, an eye-gaze input apparatus 1A in the second embodiment further includes a notification unit 60 (an example of a notification device) in addition to the configuration of the eye-gaze input apparatus 1 in the first embodiment. Also, eye-gaze input processing executed by the eye-gaze input apparatus 1A is different from the eye-gaze input processing by the eye-gaze input apparatus 1 in the first embodiment.

In the following, the eye-gaze input processing by the eye-gaze input apparatus 1A according to the present embodiment will be described with reference to FIG. 7. Note that in FIG. 7, the same reference signs are assigned to processing steps that are the same as those in the processing of FIG. 5. Since Step S11 to S15 are the same as those in the first embodiment, a description thereof is omitted.

In Step S26, the eye-gaze input apparatus 1A gives, to a user 200, notification that an output of a command from an input/output processing unit 52 to an external device 2 is stopped. Specifically, in a case where the input/output processing unit 52 stops outputting the command to the external device 2 in Step S15, the input/output processing unit 52 instructs the notification unit 60 to give notification to the user 200. Then, upon receiving the instruction, the notification unit 60 gives, to the user 200, notification that the output of the command from the input/output processing unit 52 to the external device 2 is stopped. The notification unit 60 is, for example, a speaker installed in a vehicle 100, which gives notification to the user 200 by emitting sound from the speaker. Alternatively, the notification unit 60 may be a steering wheel 130 or a seat, which gives notification to the user 200 by a vibration thereof. Furthermore, the notification unit 60 may be an HUD device 10 or a car navigation device 140, which gives notification to the user 200 by displaying a predetermined image. The predetermined image displayed by the notification unit 60 is an example of a “second image” in the present embodiment. Then, the processing proceeds to Step S27.

In Step S27, a gesture determination unit 54 determines whether the user 200 is performing a predetermined gesture. A method of determination by the gesture determination unit 54 is similar to that in Step S13. Note that a gesture different from the predetermined gesture in Step S13 is previously set as the predetermined gesture here. The predetermined gesture in Step S27 is preferably a gesture that cannot be detected in a careless state. For example, a gesture having, a larger movement or being more complicated than the predetermined gesture in Step S13 can be set as the gesture in Step S27. The complicated gesture is, for example, a gesture including a combination of more types of movements than the predetermined gesture in Step S13.

The gesture detected by a gesture detection device 30 in Step S27 is an example of a “third input” by the user 200. The third input is not limited to gesture although a gesture is detected as the third input in the present embodiment. For example, an eye gaze, voice, a touch, or the like by the user 200 may be detected as the third input. That is, a device that detects the third input is not limited to the gesture detection device 30, and a different device such as a camera, a microphone, a touch panel, or a button can be used as long as any input by the user 200 can be detected.

In response to determining that the user 200 is performing the predetermined gesture (third input) (YES in Step S27), the gesture determination unit 54 gives, to the input/output processing unit 52, information representing that the user 200 is performing the predetermined gesture (third input), and the processing proceeds to Step S28. The input/output processing unit 52 outputs a command to an external device 2 corresponding to an input element selected by the user 200 (Step S28). Then, the processing returns to Step S12, and the processing in and after Step S12 is executed.

On the other hand, in response to determining that the user 200 is not performing the predetermined gesture (third input) (NO in Step S27), the gesture determination unit 54 gives, to the input/output processing unit 52, information representing that the user 200 is not performing the predetermined gesture (third input). Then, the processing proceeds to Step S29. In Step S29, the input/output processing unit 52 discards information from an eye-gaze determination unit 53, which is related to an input element corresponding to a gaze point of the user 200. Note that the input/output processing unit 52 may stop an input of the gaze point from an eye-gaze detection device 20 to the input/output processing unit 52 instead of or in addition to discarding the information related to the input element corresponding to the gaze point of the user 200. Then, the processing returns to Step S12, and the processing in and after Step S12 is executed.

Also, in response to determining that the user 200 is not in a careless state (NO in Step S14), the careless-state determination unit 55 gives, to the input/output processing unit 52, information representing that the user 200 is not in the careless state, and the processing proceeds to Step S28 described above.

As described above, in the present embodiment, the eye-gaze input apparatus 1A includes the notification unit 60 (notification device) in addition to the HUD device 10, the eye-gaze detection device 20, the gesture detection device 30, the state detection device 40, and the input/output device 50. In a case where it is determined that the user 200 is in the careless state, the eye-gaze input apparatus 1A gives notification to the user 200. In a case where the user 200 is not in the careless state, the eye-gaze input apparatus 1A treats an eye-gaze input by the user 200 as valid when the user 200 performs a predetermined gesture in response to the notification from the eye-gaze input apparatus 1A (notification unit 60).

Therefore, even when the user 200 is determined to be in the careless state, the eye-gaze input by the user 200 can be made valid by that the user 200 indicates predetermined intention, so that the eye-gaze input apparatus that further conforms to the intention of the user 200 can be implemented.

### Modification of Embodiments

In the foregoing first and second embodiments, images of three input elements are displayed in a row as illustrated in FIGS. 4A and 4B. Alternatively, images of four or more input elements may be displayed in a row, or images of all input elements may be displayed in a row. Furthermore, images of input elements may be displayed in a format other than a row, such as a format of a table having rows and columns. Also, the number of images of input elements to be displayed may be one or two.

Order of the steps in the flowcharts of FIGS. 5 and 7 is not limited to the illustrated order. For example, in FIGS. 5 and 7, determination of a careless state (Step S14) is performed after a determination as to whether a gesture is detected (Step S13), whereas this determination may be performed after a determination of a gaze point (Step S12). In this case, a determination as to whether a gesture (second input) is detected may not be performed. Specifically, when the eye-gaze input apparatus 1 (1A) receives an input by an eye gaze of the user 200 and the careless-state determination unit 55 determines that the user 200 is not in the careless stale, a command to the external device 2 is output. Thus, a configuration according to the present disclosure can be also adopted in an eye-gaze input apparatus including no gesture detection device 30.

In FIG. 7, in a case where a gesture (third input) by the user 200 is detected (YES in Step S27) after the notification from the notification unit 60 to the user 200 is performed (Step S26), the input/output processing unit 52 gives a command to the external device 2 (Step S28). Alternatively, the input/output processing unit 52 may be configured to be able to give a command to the external device 2 regardless of presence/absence of the notification from the notification unit 60 to the user 200. That is, even in a case where the careless-state determination unit 55 determines that the user 200 is in the careless state, the input/output processing unit 52 outputs a command to the external device 2 in a case where the gesture determination unit 54 determines that the user 200 is performing the predetermined gesture (third input) regardless of whether the notification unit 60 gives notification to the user 200. Thus, even in a case where the careless-state determination unit 55 erroneously determines that the user 200 is in the careless state, an eye-gaze input by the user 200 can be treated as valid.

In the foregoing first and second embodiments, an eye-gaze input is enabled only when a vehicle is stopped. However, the eye-gaze input may be enabled under a certain condition or without a particular condition even when the vehicle is not stopped. For example, in a case where automatic driving is performed in a vehicle capable of the automatic driving, an eye-gaze input can be enabled even when the vehicle is not stopped.

The configuration of the eye-gaze input apparatus 1 is not limited to the examples illustrated in FIGS. 1 and 6. For example, the eye-gaze input apparatus 1 may not include some of or all the HUD device 10, the eye-gaze detection device 20, the gesture detection device 30, and the state detection device 40. For example, the eye-gaze input apparatus 1 may acquire a state of the user 200 from a state detection device 40 that is an external device. Also, the video signal output unit 51, the input/output processing unit 52, the eye-gaze determination unit 53, the gesture determination unit 54, and the careless-state determination unit 55 illustrated as the functions of the input/output device 50 in FIGS. 1 and 6 may be executed as functions of a device other than the input/output device 50. For example, a state detection device 40 may have the function of the careless-state determination unit 55.

Furthermore, embodiments of the present disclosure are not limited to the above-described embodiments, and various modifications can be made within the spirit and scope of the present disclosure.

Conclusion

As is apparent from the above embodiments, the present disclosure includes the following aspects. In the following, reference signs are given in parentheses only to clearly indicate correspondence with the embodiments.

An eye-gaze input apparatus (1 or 1A) of a first aspect includes an eye-gaze determination unit (53), an input/output processing unit (52), and a careless-state determination unit (55). The eye-gaze determination unit (53) determines a first input by an eye gaze of a user (200) with respect to one or more first images of one or more input elements, the one or more first images being displayed in a display region set in front of a windshield (110) and in front of a driver seat of a vehicle (100). The input/output processing unit (52) outputs a command to an external device (2) corresponding to an input element on which the first input is detected among the one or more input elements. The careless-state determination unit (55) determines, based on a state of the user (200), whether the user (200) is in a careless state. The input/output processing unit (52) confirms the first input with respect to the input element on which the first input is detected in a case where the eye-gaze determination unit (53) determines that there is the first input on any of the one or more first images and the careless-state determination unit (55) determines that the user (200) is not in the careless state. The input/output processing unit (52) does not confirm the first input with respect to the input element in a case where the careless-state determination unit (55) determines that the user (200) is in the careless state. The first aspect is capable of restraining an unintended eye-gaze input of a case where the user (200) is in the careless stale. Thus, the eye-gaze input apparatus conforming to intention of the user (200) can be provided.

An eye-gaze input apparatus (1 or 1A) of a second aspect can be implemented by a combination with the first aspect. In the second aspect, the eye-gaze input apparatus (1 or 1A) further includes an input determination unit (54) to determine a second input by the user (200). The input/output processing unit (52) confirms the first input with respect to the input element on which the first input is detected in a case where the eye-gaze determination unit (53) determines that there is the first input with respect to any of the one or more first images, the input determination unit (54) determines that there is the second input by the user (200), and the careless-state determination unit (55) determines that the user (200) is not in the careless state. The second aspect is capable of restraining an unintended eye-gaze input of a case where the user (200) is in the careless state. Thus, the eye-gaze input apparatus conforming to intention of the user (200) can be provided.

An eye-gaze input apparatus (1 or 1A) of a third aspect can be implemented by a combination with the second aspect. In the third aspect, the eye-gaze input apparatus (1 or 1A) further includes a head-up display device (10) configured to display a first image in a display region. The head-up display device (10) displays the one or more first images in a row in the display region while not displaying an outer portion of a first image displayed at an end of the display region. The input/output processing unit (52) confirms the first input with respect to an input element at the center in a case where the eye-gaze determination unit (53) determines that there is the first input on a first image arranged at a center of the row, the input determination unit (54) determines that there is the second input by the user (200), and the careless-state determination unit (55) determines that the user (200) is not in the careless state. The third aspect is capable of showing the user (200) an image of an input element as if this image is continued to the outside of the display region, and capable of making the user (200) recognize that there is an image of an input element that is not displayed in the display region.

An eye-gaze input apparatus (1 or 1A) of a fourth aspect can be implemented by a combination with the second or third aspect. In the fourth aspect, the eye-gaze input apparatus (1 or 1A) further includes an input/output device (50), an eye-gaze detection device (20), an input detection device (30), and a state detection device (40). The input/output device (50) includes the eye-gaze determination unit (53), the input/output processing unit (52), the careless-state determination unit (55), and the input determination unit (54). The eye-gaze detection device (20) detects the first input by the eye gaze of the user (200) seated in the driver seat. The input detection device (30) detects the second input by the user (200). The state detection device (40) detects a state of the user (200). The eye-gaze determination unit (53) determines, based on a detection result acquired from the eye-gaze detection device (20), a first image out of the one or more first images, on which the first input is performed. The input determination unit (54) determines whether there is the second input by the user (200) based on a detection result acquired from the input detection device (30). The careless-state determination unit (55) determines whether the user (200) is in the careless state based on the state of the user (200) acquired from the state detection device (40). According to the fourth aspect, by performing the second input, the user (200) can confirm the input of the input element selected by the eye gaze. Thus, the eye-gaze input apparatus conforming to intention of the user (200) can be provided.

An eye-gaze input apparatus (1 or 1A) of a fifth aspect can be implemented by a combination e fourth aspect. In the fifth aspect, the input/output processing unit (52) stops receiving the first input from the eye-gaze detection device (20) in a case where the careless-state determination unit (55) determines that the user (200) is in the careless state. The fifth aspect is capable of preventing reception of an unintended eye-gaze input performed by the user (200) being in the careless state. Therefore, the eye-gaze input apparatus conforming to intention of the user (200) can be provided.

An eye-gaze input apparatus (1 or 1A) of a sixth aspect can be implemented by a combination with any one of the first to fifth aspects. In the sixth aspect, the input/output processing unit (52) stops outputting the command to the external device (2) in a case where the careless-state determination unit (55) determines that the user (200) is in the careless state. The sixth aspect is capable of preventing output of a command corresponding to an unintended eye-gaze input performed by the user (200) being in the careless state. Therefore, the eye-gaze input apparatus conforming to intention of the user (200) can be provided.

An eye-gaze input apparatus (1A) of a seventh aspect can be implemented by a combination with any one of the first to sixth aspects. In the seventh aspect, the eye-gaze detection device (1A) further includes a notification unit (60) configured to give notification to the user (200) in a case where the careless-state determination unit (55) determines that the user (200) is in the careless state. The seventh aspect is capable of giving the user (200) notification that the eye-gaze input is not treated as valid. Therefore, the user (200) can recognize that the eye-gaze input is not treated as valid.

An eye-gaze input apparatus (1A) of an eighth aspect can be implemented by a combination with the seventh aspect. In the eighth aspect, the notification unit (60) gives the notification to the user (200) by displaying a second image. The eighth aspect is capable of enabling the user (200) to easily recognize the notification because the notification is given by a display.

An eye-gaze input apparatus (1A) of a ninth aspect can be implemented by a combination with the seventh aspect. In the ninth aspect, the notification unit (60) gives the notification to the user (200) by emitting a sound. The ninth aspect is capable of enabling the user (200) to easily recognize the notification because the notification is given by a sound.

An eye-gaze input apparatus (1 or 1A) of a tenth aspect can be implemented by a combination with any one of the first to ninth aspects. In the tenth aspect, the input/output processing unit (52) confirms an input on an input element in a case where a third input is received from the user (200) determined to be in the careless state by the careless-state determination unit (55). According to the tenth aspect, in a case where an eye-gaze input of when determination of the careless state is performed conforms to intention of the user (200), the eye-gaze input can be treated as valid. Thus, the eye-gaze input apparatus that further conforms to the intention of the user (200) can be provided.

An eye-gaze input apparatus (1 or 1A) of an eleventh aspect can be implemented by a combination with any one of the second to fifth aspects. In the eleventh aspect, the second input is at least one of an eve gaze, voice, and gesture. According to the eleventh aspect, an eye-gaze input can be confirmed by a predetermined input that is at least one of the eye gaze, voice, and gesture, whereby the eye-gaze input apparatus that conforms to intention of the user (200) can be provided.

An eye-gaze input apparatus (1 or 1A) of a twelfth aspect can be implemented by a combination with the tenth aspect. In the twelfth aspect, the third input is at least one of an eye gaze, voice, and gesture. According to the twelfth aspect, an eye-gaze input can be treated as valid by a predetermined input that is at least one of the eye gaze, voice, and gesture. Thus, the eye-gaze input apparatus that further conforms to intention of the user (200) can be provided.

An eye-gaze input apparatus (1 or 1A) of a thirteenth aspect can be implemented by a combination with any one of the second to fifth aspects. In the thirteenth aspect, the input/output processing unit (52) confirms an input on an input element in a case where a third input is received from the user (200) who is determined to be in the careless state by the careless-state determination unit (55). The second input and the third input are each gesture. The gesture as the third input is different from the gesture as the second input. According to the thirteenth aspect, the gesture as the third input which is different from the gesture as the second input allows an eye-gaze input to be treated as valid, whereby the eye-gaze input apparatus that further conforms to intention of the user (200) can be provided.

An eye-gaze input apparatus according to the present disclosure is capable of restraining an unintended eye-gaze input performed by a user who is in a careless state.

What is claimed is:

1. An eye-gaze input apparatus comprising
a hardware processor configured to function as:
an eye-gaze determination unit to determine a first input by an eye gaze of a user with respect to one or more first images of one or more input elements, the one or more first images being displayed in a display region set in front of a windshield and in front of a driver seat of a vehicle;
an input/output processing unit to output a command to an external device corresponding to an input element on which the first input is detected among the one or more input elements; and
a careless-state determination unit to determine, based on a state of the user, whether the user is in a careless state,
wherein the input/output processing unit
confirms the first input with respect to the input element on which the first input is detected in a case where the eye-gaze determination unit determines that there is the first input on any of the one or more first images and the careless-state determination unit determines that the user is not in the careless state, and
does not confirm the first input with respect to the input element in a case where the careless-state determination unit determines that the user is in the careless state.

2. The eye-gaze input apparatus according to claim 1, wherein
the hardware processor is further configured to function as an input determination unit to determine a second input by the user,
wherein the input/output processing unit confirms the first input with respect to the input element on which the first input is detected in a case where
the eye-gaze determination unit determines that there is the first input on any of the one or more first images,
the input determination unit determines that there is the second input by the user, and
the careless-state determination unit determines that the user is not in the careless state.

3. The eye-gaze input apparatus according to claim 2, further comprising a head-up display device configured to display the first image in the display region, wherein
the head-up display device displays the one or more first images in a row in the display region while not displaying an outer portion of a first image displayed at an end of the display region, and
the input/output processing unit confirms the first input with respect to an input element at a center in a case where
the eye-gaze determination unit determines that there is the first input on a first image arranged at the center of the row,
the input determination unit determines that there is the second input by the user, and
the careless-state determination unit determines that the user is not in the careless state.

4. The eye-gaze input apparatus according to claim 2, further comprising:
an input/output device including the hardware processor;
an eye-gaze detection device configured to detect the first input by the eye gaze of the user seated in the driver seat;
an input detection device configured to detect the second input by the user; and
a state detection device configured to detect a state of the user, wherein
the eye-gaze determination unit determines, based on a detection result acquired from the eye-gaze detection device, a first image out of the one or more first images, on which the first input is performed,
the input determination unit determines whether there is the second input by the user based on a detection result acquired from the input detection device, and
the careless-state determination unit determines whether the user is in the careless state based on the state of the user acquired from the state detection device.

5. The eye-gaze input apparatus according to claim 4, wherein the input/output processing unit stops receiving the first input from the eye-gaze detection device in a case where the careless-state determination unit determines that the user is in the careless state.

6. The eye-gaze input apparatus according to claim 1, wherein the input/output processing unit stops outputting the command to the external device in a case where the careless-state determination unit determines that the user is in the careless state.

7. The eye-gaze input apparatus according to claim 1, further comprising a notification device configured to give notification to the user in a case where the careless-state determination unit determines that the user is in the careless state.

8. The eye-gaze input apparatus according to claim 7, wherein the notification device gives the notification to the user by displaying a second image.

9. The eye-gaze input apparatus according to claim 7, wherein the notification device gives the notification to the user by emitting a sound.

10. The eye-gaze input apparatus according to claim 11, wherein the input/output processing unit confirms the input on the input element in a case of receiving a third input from the user who is determined to be in the careless state by the careless-state determination unit.

11. The eye-gaze input apparatus according to claim 2, wherein the second input is at least one of an eve gaze, voice, and gesture.

12. The eye-gaze input apparatus according to claim 10, wherein the third input is at least one of an eye gaze, voice, and gesture.

13. The eye-gaze input apparatus according to claim 2, wherein
the input/output processing unit confirms the input on the input element in a case of receiving a third input from the user who is determined to be in the careless state by the careless-state determination unit,
the second input and the third input are each gesture, and
a gesture as the third input is different from a gesture as the second input.
